(19) United States
(12) Patent Application Publication
(10) Pub. No.: US 2007/0042478 A1
(43) Pub. Date: Feb. 22, 2007

(54) Fermentation of monovalent secondary alcohols in order to form corresponding ketones

(75) Inventor: Jurgen Rabenhorst, Hoxter (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: Symrise GmbH & Co. KG, Holzminden (DE)

(21) Appl. No.: 10/596,053

(22) PCT Filed: Nov. 24, 2004
(86) PCT No.: PCT/EP04/53076

§ 371 Date: May 26, 2006

(30) Foreign Application Priority Data
Nov. 28, 2003 (DE) 103 55 734.2

(51) Int. Cl. C12P 7/26; C12N 1/20

(52) U.S. Cl. 435/148; 435/252.3

(57) ABSTRACT

The present invention concerns a fermentation process for converting a monohydric secondary alcohol having 5 or more carbon atoms into the corresponding ketone, suitable microorganisms for that purpose.

The present invention concerns a fermentation process for converting a monohydric secondary alcohol having 5 or more carbon atoms into the corresponding ketone, suitable microorganisms for that purpose and enzymes thereof. Of particular interest here is the production of aliphatic ketones from the corresponding aliphatic monohydric secondary alcohols, in particular the production of pentan-2-one from 2-pentanol, heptan-2-one from 2-heptanol, octan-2-one from 2-octanol, nonan-2-one from 2-nonanol, 1-penten-3-one from 1-penten-3-ol, 1-hexen-3-one from 1-hexen-3-ol, hexan-3-one from 3-hexanol, heptan-3-one from 3-heptanol and octan-3-one from 3-octanol.

The oxidation of primary alcohols to form the corresponding acids is known from the literature, as the publications EP 289 822, DE 195 03 598, J. Chem. Tech. Biotechnol. 1997, 68, 214-218, and J. Chem. Tech. Biotechnol. 1997, 70, 294-298, for example, confirm. Lett. Appl. Microbiol. 1995, 20, 365-368 describes the conversion of various alcohols to acids and 2-butanone and mentions the more strongly inhibiting action of 2-butanone in comparison to the acids. The asymmetrical reduction of ketones to produce enantiomer-pure alcohols with Gluconobacter oxydans is also known (Adlerkreuz, Enzyme Microb. Technol. 1991, 13, 9-14). The oxidation of secondary alcohols with alcohol oxidases of various hydrocarbon-degrading yeasts is known (Appl. Microbiol. Biotechnol. 1992, 37, 66-73; Tetrahedron Asymmetry 2000, 11, 2367-2373). This reaction proceeds with high stereoselectivity, such that only a maximum of 50 percent of the substrate is converted, limiting the possible product yield. Moreover, NAD is required as a cofactor. NAD must therefore either be added in equimolar amounts, which on economic grounds is not possible, or the NADH that is formed must be regenerated by means of a second enzymatic reaction, which is likewise associated with time and costs. Finally, Adachi et al., Appl. Microbiol Biotechnol. (2003) 60:643-653, describe the oxidation of polyhydric alcohols to form molecules having both hydroxy and keto functions.

The previous fermentation processes commonly achieve only undesirably low product yields. Enzymatic processes, such as those described by Adlerkreuz (loc. cit.), also require the regeneration of coenzymes needed to produce the ketones, which makes enzymatic processes more difficult and costly to perform.

The object of the present invention was therefore to provide a process by means of which monohydric secondary alcohols having 5 or more carbon atoms can be converted into the corresponding ketones. The process should be as simple as possible to manage, allow good product yields in a short reaction time, and be able to be performed at a reasonable cost. Means for performing these processes should also be provided. In particular, the process to be provided and the means suitable for its performance should allow the conversion of an aliphatic monohydric secondary alcohol to the corresponding ketone.

A process is therefore provided for converting a monohydric secondary alcohol having 5 or more carbon atoms to the corresponding ketone, wherein the process comprises fermentation of the alcohol to form the ketone using a bacterium of the Gluconobacter and/or Acetobacter genus in a fermentation medium.

The sought-after advantages can be achieved using this process. In particular, the conversion of a straight-chain monohydric secondary alcohol to the corresponding ketone proceeds easily, in a good yield, reproducibly and at a reasonable cost.

Furthermore, several of the monohydric secondary alcohols can be fermented to form the corresponding ketones simultaneously.

It is particularly advantageous that the conversion of the alcohol to the ketone is performed in a fermentation process, in other words using whole cells of the bacterium used for fermentation. Optimum results can be obtained here if the fermentation is performed using a bacterium of the cited genera that is capable of multiplication. A bacterium is deemed to be capable of multiplication if, after transfer to a conventional cultivation medium (e.g. DSM Medium 105: glucose 100 g; yeast extract 10 g; CaCO₃ 20.0 g; distilled water 1000 ml adjusted to pH 6.8) and cultivation under conventional cultivation conditions (e.g. 25° C.), its cell count doubles within 24 hours, wherein a minimum cell concentration may have to be maintained at the start of cultivation. A particular advantage of this is that regeneration of coenzymes and cofactors can be dispensed with, which makes the performance of the process considerably simpler. In preferred embodiments, in the process according to the invention a monohydric secondary alcohol having 5 or more carbon atoms, but at most 20 and particularly preferably at most 14 carbon atoms, is fermented to form the corresponding ketone using a bacterium of the genus Gluconobacter and/or Acetobacter. The particularly preferred embodiments of the invention and of the strains according to the invention specified below relate in particular also to the fermentation of monohydric secondary alcohols having the aforementioned preferred maximum numbers of carbon atoms.

Fermentation of a monohydric secondary alcohol having 5 or more carbon atoms (preferably having a maximum of 20 carbon atoms and particularly preferably having a maximum of 14 carbon atoms) preferably takes place using a bacterium of the Gluconobacter genus, particularly good product yields being obtained above all in fermentations using bacteria of the species Gluconobacter oxydans. Particularly good results can be achieved if fermentation is brought about with the aid of a bacterium of the strain Gluconobacter oxydans ssp. suboxydans DSM 12884. The use of this strain for the fermentation of a monohydric secondary alcohol, in particular an aliphatic and preferably straight-chain alcohol, having 5 or more carbon atoms, preferably at most 20 carbon atoms and particularly preferably at most 14 carbon atoms, to form the corresponding ketone, is therefore particularly preferred. This strain is capable of fermenting at least 1 g/l of 1-penten-3-one within 48 hours when fermented in DSM Medium 105 containing 3 g/l of 1-penten-3-ol at 25° C.

Fermentation is conveniently performed with a pure culture of the cited bacteria, in particular Gluconobacter sp. DSM 12884.

Synthetic, semisynthetic or complex media can be used as the nutrient medium for the organisms used according to the invention. In particular, the nutrient media can be used as the fermentation medium and can contain carbon-containing and nitrogen-containing compounds, inorganic salts, optionally trace elements and vitamins.

Carbohydrates, hydrocarbons or basic organic chemicals can be used as carbon-containing compounds. Examples of compounds that can preferably be used are sugars, alcohols or sugar alcohols, organic acids or complex mixtures. A preferred sugar alcohol is mannitol.

Citric acid or acetic acid can preferably be used as organic acids. Malt extract, yeast extract, casein or casein hydrolysate are preferred in particular as constituents of semisynthetic or complex media.

Inorganic compounds, for example nitrates and ammonium salts, can be used in particular as nitrogen-containing substrates. Equally, organic nitrogen sources, such as yeast extract, soya flour, cottonseed flour, wheat gluten, casein, casein hydrolysate and maize steep liquor, can be used.

The inorganic salts that can be used include, for example, sulfates, nitrates, chlorides, carbonates and phosphates. As metals the cited salts preferably contain sodium, potassium, magnesium, manganese, calcium, zinc and/or iron.

It is also preferable, prior to fermentation, to precultivate a culture of the bacterium used for fermentation in a cultivation medium which contains mannitol, malt extract, yeast extract, soya flour, cottonseed flour, wheat gluten, casein, casein hydrolysate, maize steep liquor, citric acid, acetic acid or mixtures of two or more of these constituents and which has a pH of 4 to 8 at the start of precultivation. The cultivation facilitates fermentation, particularly if bacteria are to be used which before the start of fermentation are in a state of rest, particularly in a glycerol culture. It is likewise possible firstly to precultivate the bacteria used for fermentation in a cultivation medium and after precultivation to add the alcohol(s) to be fermented to this medium. A medium consisting of 1-2 g of D-mannitol and 1-2 g of yeast extract, based on 100 ml of medium, at a pH of 5-6, has proved particularly suitable as a cultivation medium.

The temperature for fermentation and cultivation is preferably in the range from 10 to 40° C. The range from 20 to 35° C. is particularly preferred, 25 to 27° C. being highly preferred, since at these temperatures particularly good conversions and product yields were able to be achieved.

At the start of fermentation and at the start of cultivation respectively, the pH of the fermentation and cultivation medium is preferably 4 to 8, the range from 4.8 to 6.3 being particularly preferred.

Particularly good fermentation results were able to be obtained if at the time of substrate addition the optical density of the culture is at least OD₆₀₀ 2.0, the live bacterial count is at least 1×10⁸/ml and the concentration of dissolved oxygen in the fermentation medium before substrate addition is no more than 10%.

All bioreactors known to the person skilled in the art can be used in principle to perform the process according to the invention. A bioreactor that is suitable for performing submerged fermentation processes is preferably used for fermentation. This means that bioreactors can be used according to the invention with or without mechanical mixing equipment. The former include, for example, shaking apparatus, bubble column reactors or loop reactors. The latter include bioreactors with any form of stirrer.

The process according to the invention can be performed continuously or batchwise. The period of fermentation until a maximum amount of product is obtained depends on the specific nature of the organism used. Particularly good product yields were able to be obtained with a fermentation of 2 to 200 hours, fermentation preferably being performed for 8 to 78 hours.

The following ketones in particular can be produced with the process according to the invention: pentan-2-one, heptan-2-one, octan-2-one, nonan-2-one, 1-penten-3-one, 1-hexen-3-one, hexan-3-one, heptan-3-one and octan-3-one.

The invention is illustrated in more detail using the examples below, wherein the examples are not to be understood as limiting the subject of the invention:

### EXAMPLE 1

### Production of the Preculture

A 500 ml Erlenmeyer flask with side arm containing 100 ml of sterile culture medium, consisting of 1.25 g of D-mannitol and 1.25 g of yeast extract at pH 5.6, is inoculated with 0.9 ml of a glycerol culture of Gluconobacter sp. DSM 12884. The flask is incubated for 16 hours in a rotary shaking machine at 25° C. and 100 rpm.

### EXAMPLE 2

### Production of Natural 1-penten-3-one from 1-penten-3-ol

125 g of mannitol and 125 g of yeast extract are dissolved in 9.9 l of water in a 10 l fermenter, 10 ml of antifoam agent are added and the pH adjusted to 6.3. The fermentation medium produced in this way is sterilised for 30 minutes at 121° C. After cooling to 25° C., the fermenter is inoculated with the preculture from Example 1.

The fermentation medium is stirred during the reaction of 1-penten-3-ol and a preceding growth phase. The speed of the stirrer is 500 rpm, the inlet air supply rate 5 Nl/min; the temperature 25° C. After establishing these parameters, the fermentation medium is inoculated with the 100 ml of preculture according to Example 1.

After a cultivation time (growth phase) of about 23 hours, the substrate 1-penten-3-ol (150 ml) is added to the fermenter through a funnel and fermentation is begun. At the same time the inlet air supply is throttled down from 5 Nl/min to 1 Nl/min (□0.1 vvm). The pH remains relatively stable during the cultivation time and fermentation.

Since both the substrate 1-penten-3-ol and the product 1-penten-3-one are very highly volatile, the outlet air from the fermenter is passed through an active condenser and then collected in a cold trap which is cooled with a mixture of isopropanol and dry ice.

Fermentation is terminated after approximately 70 hours. According to HPLC, the final concentration of 1-penten-3-one in the fermentation medium is approx. 5 g/l. The substrate 1-penten-3-ol is still present in the fermentation medium in a concentration of about 2 g/l. At the end of the process the cold trap contains approx. 20 g of 1-penten-3-one.

### EXAMPLE 3

### Production of 2-pentanone from 2-pentanol

Two 100 ml Erlenmeyer flasks with ground glass stoppers, each containing 20 ml of sterile cultivation medium, consisting of 0.25 g of D-mannitol and 0.25 g of yeast extract at pH 5.6, are inoculated with 200 μl of a preculture of Gluconobacter sp. DSM 12884.

The flasks are incubated for 20 hours in a rotary shaking machine at 25° C. and 100 rpm. During this growth phase the flasks are sealed aseptically with cotton plugs.

200 μl of 2-pentanol are then added to each flask, the flasks are closed with sterile ground glass stoppers and incubated again. 24 and 48 hours after addition of the 2-pentanol, a 1 ml sample is taken, extracted with 2 ml of hexane and analysed by gas chromatography. After 24 hours, 83% (percent per unit area) of 2-pentanone and 10.5% of 2-pentanol were found. After 48 hours the content of 2-pentanone is over 92%.

### EXAMPLE 4

### Production of 2-heptanone from 2-heptanol

Two 100 ml Erlenmeyer flasks with ground glass stoppers, each containing 20 ml of sterile cultivation medium, consisting of 0.25 g of D-mannitol and 0.25 g of yeast extract at pH 5.6, are inoculated with 200 μl of a preculture of Gluconobacter sp. DSM 12884.

The flasks are incubated for 20 hours in a rotary shaking machine at 25° C. and 100 rpm. During this growth phase the flasks are sealed aseptically with cotton plugs.

100 μl of 2-heptanol are then added to each flask, the flasks are closed with sterile ground glass stoppers and incubated again. 24 and 48 hours after addition of the 2-heptanol, a 1 ml sample is taken, extracted with 2 ml of hexane and analysed by gas chromatography. After 24 hours, 54.7% (percent per unit area) of 2-heptanone and 35% of 2-heptanol were found. After 48 hours the content of 2-heptanone is over 59%, whilst 31.9% of 2-heptanol are still present.

### EXAMPLE 5

### Production of 2-octanone from 2-octanol

Two 100 ml Erlenmeyer flasks with ground glass stoppers, each containing 20 ml of sterile cultivation medium, consisting of 0.25 g of D-mannitol and 0.25 g of yeast extract at pH 5.6, are inoculated with 200 μl of a preculture of Gluconobacter sp. DSM 12884.

The flasks are incubated for 20 hours in a rotary shaking machine at 25° C. and 100 rpm.

During this growth phase the flasks are sealed aseptically with cotton plugs.

100 μl of 2-octanol are then added to each flask, the flasks are closed with sterile ground glass stoppers and incubated again. 24 hours after addition of the 2-octanol, a 1 ml sample is taken, extracted with 2 ml of hexane and analysed by gas chromatography. 37.2% (percent per unit area) of 2-octanone and 46.1% of 2-octanol were found.

### EXAMPLE 6

### Production of 2-nonanone from 2-nonanol

Two 100 ml Erlenmeyer flasks with ground glass stoppers, each containing 20 ml of sterile cultivation medium, consisting of 0.25 g of D-mannitol and 0.25 g of yeast extract at pH 5.6, are inoculated with 200 μl of a preculture of Gluconobacter sp. DSM 12884.

The flasks are incubated for 20 hours in a rotary shaking machine at 25° C. and 100 rpm. During this growth phase the flasks are sealed aseptically with cotton plugs.

100 μl of 2-nonanol are then added to each flask, the flasks are closed with sterile ground glass stoppers and incubated again. 48 hours after addition of the 2-nonanol, a 1 ml sample is taken, extracted with 2 ml of hexane and analysed by gas chromatography. 32% (percent per unit area) of 2-nonanone and 48.8% of 2-nonanol were found.

### EXAMPLE 7

### Production of 3-octanone from 3-octanol

Two 100 ml Erlenmeyer flasks with ground glass stoppers, each containing 20 ml of sterile cultivation medium, consisting of 0.25 g of D-mannitol and 0.25 g of yeast extract at pH 5.6, are inoculated with 200 μl of a preculture of Gluconobacter sp. DSM 12884. The flasks are incubated for 20 hours in a rotary shaking machine at 25° C. and 100 rpm. During this growth phase the flasks are sealed aseptically with cotton plugs.

100 μl of 3-octanol are then added to each flask, the flasks are closed with sterile ground glass stoppers and incubated again. 72 hours after addition of the 3-octanol, a 1 ml sample is taken, extracted with 2 ml of hexane and analysed by gas chromatography. After 72 hours, 4.4% (percent per unit area) of 3-octanone and 81.5% of 3-octanol were found.

### EXAMPLE 8

### Production of 3-hexanone from 3-hexanol

Two 100 ml Erlenmeyer flasks with ground glass stoppers, each containing 20 ml of sterile cultivation medium, consisting of 0.25 g of D-mannitol and 0.25 g of yeast extract at pH 5.6, are inoculated with 200 μl of a preculture of Gluconobacter sp. DSM 12884.

The flasks are incubated for 20 hours in a rotary shaking machine at 25° C. and 100 rpm. During this growth phase the flasks are sealed aseptically with cotton plugs.

200 μl of 3-hexanol are then added to each flask, the flasks are closed with sterile ground glass stoppers and incubated again. 24 and 54 hours after addition of the 3-hexanol, a 1 ml sample is taken, extracted with 2 ml of hexane and analysed by gas chromatography. After 24 hours, 70.7% (percent per unit area) of 3-hexanone and 24.5% of 3-hexanol were found. After 54 hours the content of 3-hexanone is over 88%, whilst only 7.4% of the substrate are still present.

### EXAMPLE 9

### Production of 1-hexen-3-one from 1-hexen-3-ol

Two 100 ml Erlenmeyer flasks with ground glass stoppers, each containing 20 ml of sterile cultivation medium, consisting of 0.25 g of D-mannitol and 0.25 g of yeast extract at pH 5.6, are inoculated with 200 μl of a preculture of Gluconobacter sp. DSM 12884.

The flasks are incubated for 20 hours in a rotary shaking machine at 25° C. and 100 rpm. During this growth phase the flasks are sealed aseptically with cotton plugs.

200 μl of 1-hexen-3-ol are then added to each flask, the flasks are closed with sterile ground glass stoppers and incubated again. 8 hours after addition of the 2-hexen-2-ol, a 1 ml sample is taken, extracted with 2 ml of hexane and analysed by gas chromatography. 25.4% (percent per unit area) of 1-hexen-3-one and 69.7% of 1-hexen-3-ol were found.

### EXAMPLE 10

### Production of 3-heptanone from 3-heptanol

Two 100 ml Erlenmeyer flasks with ground glass stoppers, each containing 20 ml of sterile cultivation medium, consisting of 0.25 g of D-mannitol and 0.25 g of yeast extract at pH 5.6, are inoculated with 200 μl of a preculture of Gluconobacter sp. DSM 12884.

The flasks are incubated for 20 hours in a rotary shaking machine at 25° C. and 100 rpm.

During this growth phase the flasks are sealed aseptically with cotton plugs.

200 μl of 3-heptanol are then added to each flask, the flasks are closed with sterile ground glass stoppers and incubated again. 8 hours after addition of the 3-heptanol, a 1 ml sample is taken, extracted with 2 ml of hexane and analysed by gas chromatography. 2.3% (percent per unit area) of 3-heptanone and 92.2% of 3-heptanol were found.

1. Process for converting a monohydric secondary alcohol having 5 or more carbon atoms to the corresponding ketone, comprising converting said alcohol to form said ketone by fermenting said alcohol using a bacterium of the Gluconobacter and/or Acetobacter genus in a fermentation medium.

2. Process according to claim 1, characterised in that the fermenting brought about using a bacterium of the Gluconobacter genus.

3. Process according to claim 2, characterised in that the fermenting is brought about using a bacterium of the strain Gluconobacter sp. DSM 12884.

4. Process according to claim 1, characterised in that the fermentation medium contains mannitol, malt extract, yeast extract, soya flour, cottonseed flour, wheat gluten, casein, casein hydrolysate, maize steep liquor, citric acid, acetic acid or mixtures or several of these constituents and has a pH of 4 to 8 at the start of fermentation.

5. Process according to claim 1, characterised in that before fermentation, the bacterium used for fermentation is precultivated in a cultivation medium which contains mannitol, malt extract, yeast extract, soya flour, cottonseed flour, wheat gluten, casein, casein hydrolysate, maize steep liquor, citric acid, acetic acid or mixtures of two or more of these constituents and has a pH of 4 to 8 at the start of precultivation.

6. Process according to claim 1, characterised in that fermentation takes place at a temperature of 20 to 40° C.

7. Process according to claim 1, characterised in that the dissolved oxygen concentration in the fermentation medium is less than or equal to 5%.

8. Process according to claim 1, characterised in that in the fermentation 2-pentanol is converted to pentan-2-one, 2-heptanol to heptan-2-one, 2-octanol to octan-2-one, 2-nonanol to nonan-2-one, 1-penten-3-ol to 1-penten-3-one, 1-hexen-3-ol to 1-hexen-3-one, 3-hexanol to hexan-3-one, 3-heptanol to heptan-3-one and/or 3-octanol to octan-3-one.

9. Gluconobacter sp. DSM 12884.

10. (canceled)
